(19) United States
(12) Patent Application Publication
(10) Pub. No.: US 2004/0254512 A1
(43) Pub. Date: Dec. 16, 2004

(54) Devices for skin antisepsis during surgical procedures

(76) Inventor: Sumathi Paturu, Pleasant Grove, AL (US)

Correspondence Address: DR. SUMATHI PATURU, MD., 207, BROOKE-LYN TERRACE, PLEASANT GROVE, AL, 35127, US

(21) Appl. No.: 10/460,787

(22) Filed: Jun. 13, 2003

(51) Int. Cl. A61F 13/00

(52) U.S. Cl. 602/43; 602/42

(57) ABSTRACT

Surgical site infection is the most common cause of the nosocomial infections, and is responsible for increased morbidity, prolonged hospital stay, and expenses, and also for the development of antibiotic resistance.

There are not enough research studies done on this type of surgical infections, due to many limiting factors, but limited research data studied, indicates that most of the infection is in the superficial and deep sites of the incision, and not in the deeper visceral planes that tells, that the patient's skin, that does not have any surgical barrier, can be the source of the infection.

The devices that are described, are meant to provide the surgical barrier needed for the patient's skin, and also ways for uncontaminated surgical scrubbing during different types of surgical procedures, and these are the methods not previously described in the medical literature, that can definitely decrease the incidence of surgical sepsis in every possible way.

### INTRODUCTION

Surgical site infection (SSI) is the leading nosocomial cause of morbidity, and a source of excess medical cost. Sterile gloves contribute to preventing the surgical wound contamination, but there can be other preventable ways also that are over looked, and it could be more than the surgeons hands that can contaminate the operating field.

A recent article in JAMA (The Journal of American medical association), August 2002, volume 288, no. 6, 722-727, titled ‘Hand rubbing with an aqueous alcoholic solution vs traditional surgical hand scrubbing and 30 day surgical site infection rates, a randomized equivalence study’ done in France, in both teaching and non teaching hospitals, was very interesting. It was evident from the article - because of the need for very large population sample together with the existence of numerous confounding factors, and prohibitive costs, clinical studies, comparing the risks of nosocomial infection after different hand antisepsis protocols are scarce. The article also states, that infection control epidemiology has clearly demonstrated that bacteria responsible for SSI can be shed from the surgical team's hands, despite antisepsis. However it was not emphasized how that conclusion was derived. It is also not also not clear why when so much emphasis was given to hand washing techniques of the operating team, the incompleteness of the patient's skin sterilization was not discussed as if it is non existent problem. Patient's skin can be a source of serious MRSA infection, and there can be cases of Staph. Epedermidis infections in cardiac surgeries. In the study group, the surgical services used 2 hand cleaning methods alternatively every other month. A hand rubbing protocol with 75% aqueous alcoholic solution containing Propanol-1, Propanol-2, and Mecetronium EtilSulphate; and a hand scrubbing protocol with an antiseptic preparation containing 4% Povidone Iodine, or 4% Chlorhexidine Gluconate.

In this study 4387 patients were considered for analysis. The surveillance system identified 99 in hospital, and 9 post discharge SSIs (total of 108). In the hand scrubbing protocol—22 SSIs were superficial, 19 deep, and 8 organ space and 5 unknown. In the hand rubbing protocol 24 SSIs were superficial, 15 deep, 7 organ space and 9 unknown. The distribution of these categories of SSIs did not differ between the two protocols. If both the groups are put together, there are 45 superficial, 34 deep, 15 organ space (deepest), and 14 unknown type of infections. And 45+34 makes 79 cases of superficial and deep abdominal wall infections, and only 15 organ space (intra abdominal or peritoneal). These statistics tell that the abdominal wall infection is the major cause of the post operative morbidity and it is 6 times more than organ space i.e. intra abdominal or visceral space infection. So there is good reason to believe that the infection could be coming from the patient's skin also. If it is coming from the surgical team's hands, the infection rate should be equally prevalent in all the anatomical sites. In fact, it should be more in the viscera and the organ spaces where the direct handling with the surgeon's hands and fingers happen for a longer period of time. Most of the time, the incision site is dealt with instruments. Accidental cutting of the gloves also is more prone to occur in the deeper planes where the visibility of the site, or the space available is not as good, for the surgeon or the assistant using the scissors; for the same reason it also goes unnoticed, until the time the surgeon's hands are brought to the surface. Cut in the glove is uncommon during the times the surgical incision is given, or when it is closed, because there is direct visibility, and ample space to work on precisely. Yet the infection rate as mentioned, seems to be more in the superficial areas which tells that significant source of infection is coming from the skin of the patient, into the superficial and the immediate deeper planes of the surgical incision.

Maintaining strict perioperative antiseptic precautions are multi-factorial, the patient's skin being the most important, and the most neglected. Absence of the surgical barrier to the patient's skin, and the invariable lack of hygiene in this area postoperatively also, due to imperceptible or perceptible perspiration compounded by the lack of bath for few days, makes it the most vulnerable area as the source of infection. The surgeon's hands have a sterile surgical barrier, but the patient's skin is naked, and the very first cut of any surgery is being started on a potentially unclean area (It is like doing surgery without hand gloves). It is surprising that there are not that many surgical infections, and I am sure that credit has to be given to the body's natural defenses, or the antibiotics that many surgeons start as prophylaxis, or for a cause. The skin contamination which is the most significant problem, can be easily taken care of, to avoid the post operative infections in the majority of cases. During the surgery, the patient's skin needs a sterile surgical barrier too. The following devices that are being described are meant to accomplish that.

### BRIEF DESCRIPTION OF THE DEVICES

The surgical devices being described in this invention, are aids to achieve the best surgical barriers, to the patient's skin, that enables the maximum prevention of contamination from this vulnerable area, that is difficult or impossible to clean, especially if it happens to be the scrotal or perineal area. As the techniques involved in these surgeries are different, the devices described are different for each surgery. How ever for all the devices, the material used is sterile, water proof, adherent, Poly urethane plastic, that will be attached to the skin of the patient, acting as a fluid proof barrier on which the surgery can be done, incision being given on this plastic surface, which is like the patient's sterile second skin.

The ways of sterile water supply, and sterile hand washing in the operating room, is also described in this invention, with out which, the procedures of antisepsis are by no means complete, or meaningful.

The following embodiments of devices are being described, and claimed as new invention by the inventor.

1. Sterile surgical occlusive dressing 1 for major surgeries (SSOD-1 FMS)

2. Sterile surgical occlusive dressing 2 for major surgeries (SSOD-2 FMS)

3. Sterile surgical occlusive dressing 1 for minor and miscellaneous surgeries (SSOD-1 mini surge)

4. Sterile surgical occlusive dressing 2 for minor and miscellaneous surgeries (SSOD-2 mini surge)

5. Sterile surgical occlusive dressing for urological surgeries.

6. Sterile surgical occlusive dressing for gynecological surgeries.

7. Sterile skin edge patches.

8. Sterile transparent plastic water tanks and their accessories.

### DRAWINGS

FIG. 1A—Sterile surgical Occlusive dressing-1 for major surgeries—(SSOD-1 FMS)—The Superficial surgical surface

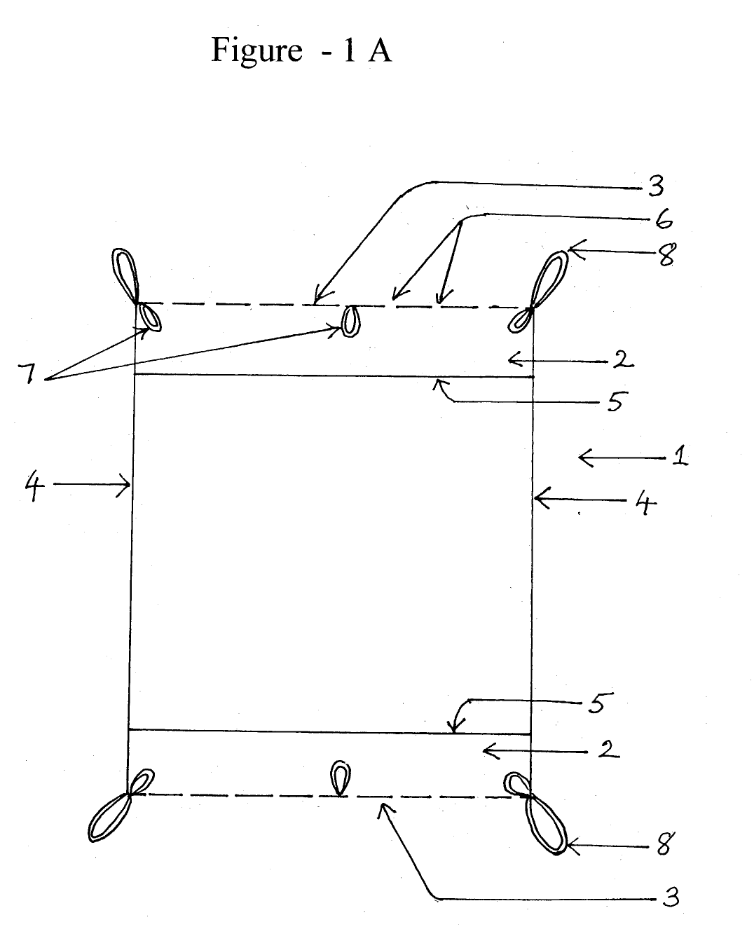

FIG. 1B—Sterile surgical Occlusive dressing-1 for major surgeries—(SSOD-1 FMS) The deep skin surface.

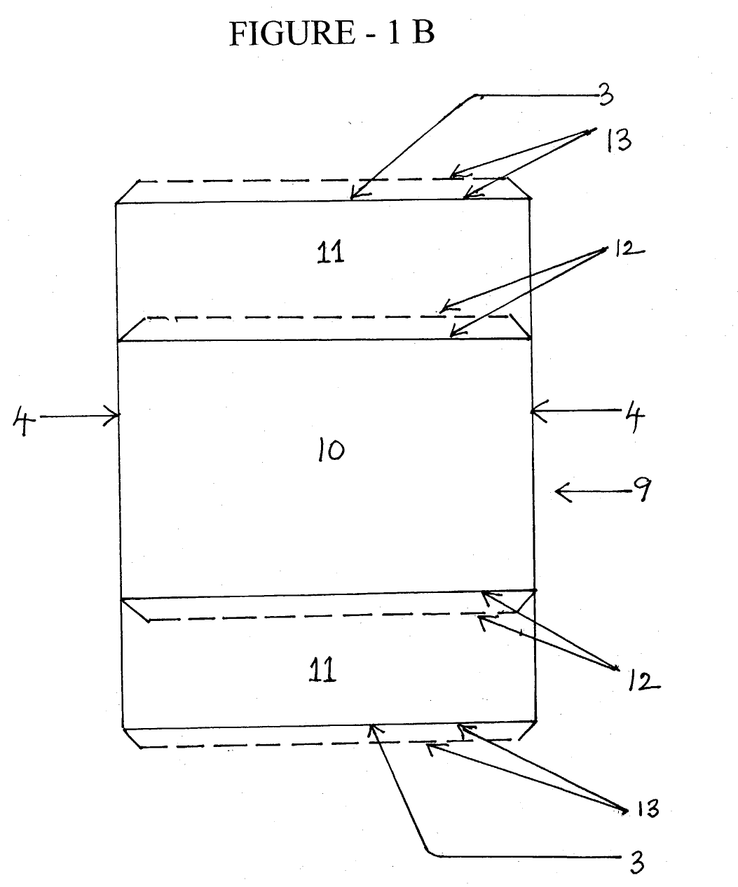

FIG. 2A—Sterile surgical Occlusive dressing-2 for major surgeries—(SSOD-2 FMS) The superficial surgical surface.

FIG. 2B—Sterile surgical occlusive dressing-2 for major surgeries—(SSOD-2 FMS) The deep skin surface.

FIG. 3A—Sterile occlusive dressing-1 for minor surgeries—(SSOD-1 mini surge)—superficial surgical surface.

FIG. 3B—Sterile occlusive dressing-1 for minor surgeries—(SSOD-1 mini surge)—the deep skin surface.

FIG. 4A—Sterile occlusive dressing-2 for minor surgeries—(SSOD-2 mini surge)—the superficial surgical surface.

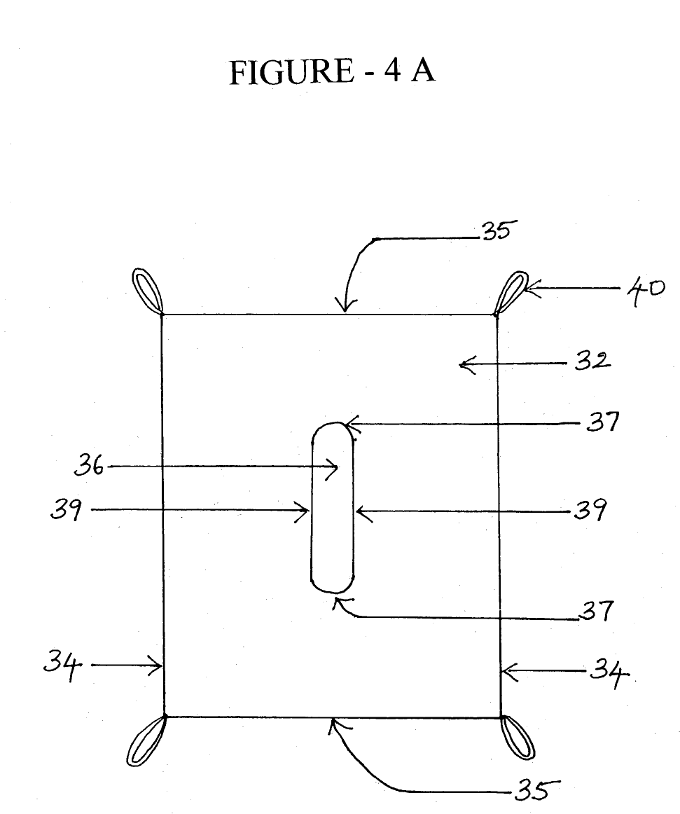

FIG. 4B—Sterile occlusive dressing-2 for minor surgeries—(SSOD-2 mini surge)—the deep skin surface.

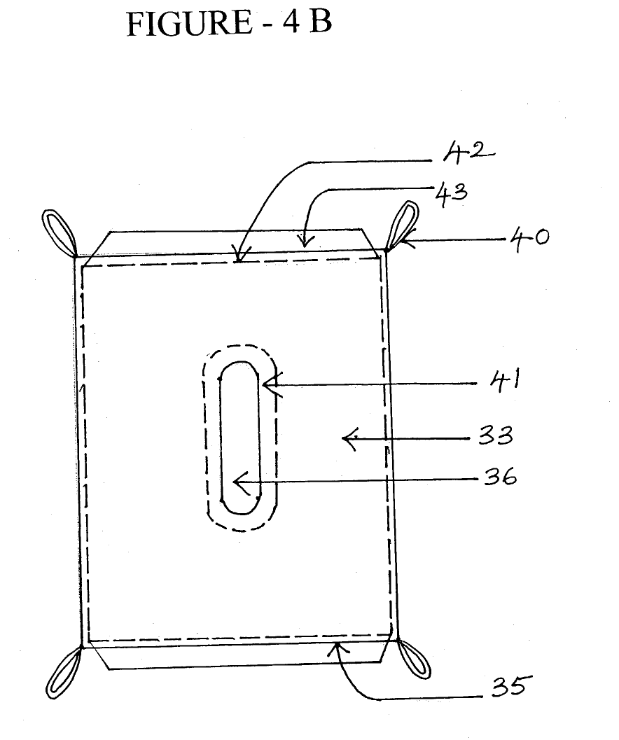

FIG. 5A—Sterile surgical occlusive dressing for urological surgeries—The superficial surgical surface

FIG. 5B—Sterile surgical occlusive dressing for urological surgeries—The deep skin surface

FIG. 6A—Sterile surgical occlusive dressing for the obstetrics/gynecological surgeries—The superficial surgical surface.

FIG. 6B—Sterile surgical occlusive dressing for the obstetrics/gynecological surgeries—the deep skin surface.

FIG. 7A—Sterile skin edge patches—attached to the skin incision.

FIG. 7B—Sterile skin edge patches—with a straight and curvilinear medial ends.

FIG. 8A—Sterile transparent plastic water tank with a cap, and without attached tap.

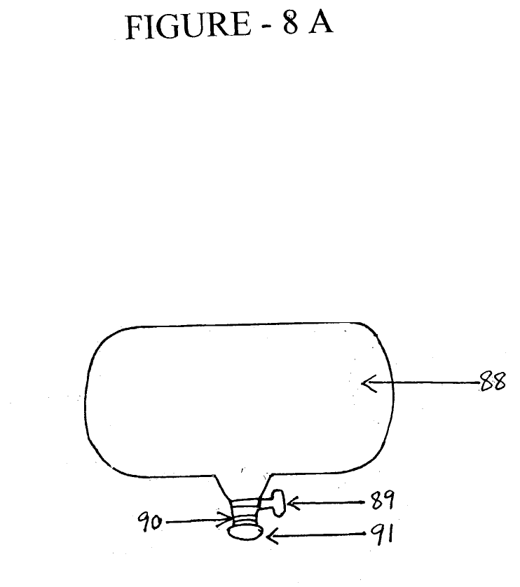

FIG. 8B—Sterile plastic tap faucet in the pouch.

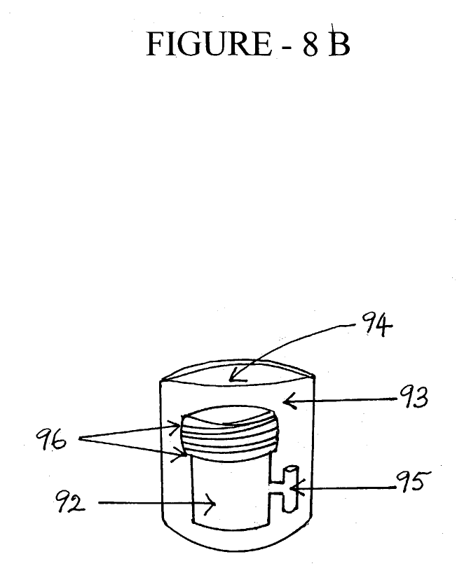

FIG. 8C—Sterile transparent water tank with the water faucet attached.

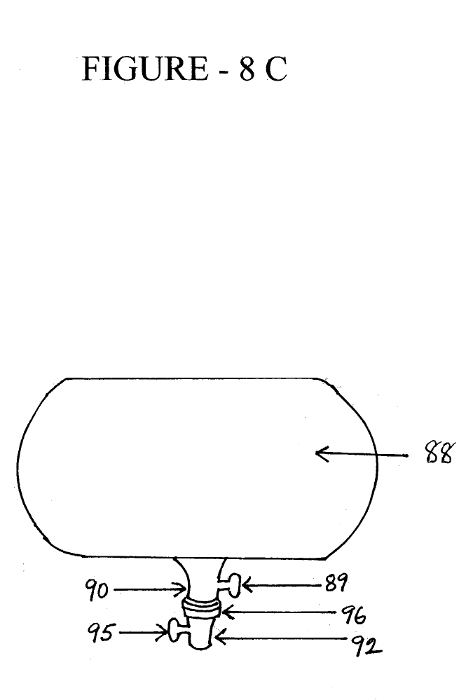

FIG. 8D—Sterile surgical site irrigating water tubing to be attached to the sterile transparent water tanks

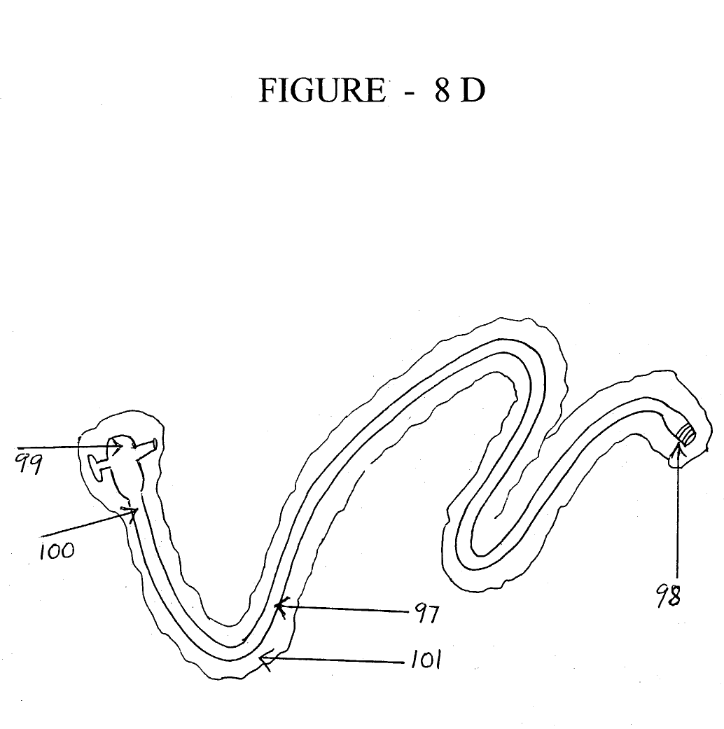

### THE DETAILED DESCRIPTION OF THE INVENTION

The Sterile Surgical Occlusive Dressing-1 for Major Surgeries (SSOD-1 FMS)

In the following structural description of the device, the way it is used is also described simultaneously at some places, because it is better understood that way.

The term major surgery in this contest only implies to the length of the incision involved, and not the quality of the surgery, like it is used in the standard terminology.

The sterile surgical occlusive dressing-1 for major surgeries (SSOD-1 FMS) can be used for Thoracoabdominal, pelvic, and back surgeries. It is made up of 15-25″×20-30″ size, rectangular shaped, sterile, transparent, waterproof, polyurethane adhesive occlusive dressing. Different sizes ranges are available as mentioned, for different patient sizes, with a size difference of 2″ and are suitable to place any where on the Thorax, abdomen or the back. When secured on to the skin surface, it is like the patient's sterile second skin, on which the incision can be given, with out the knife or the surgeon's gloves carrying the skin bacteria into the deeper planes of the surgical site. The dressing is water resistant, not to allow blood or water to seep through, in between the skin and the dressing. This water resistant occlusive sterile surgical barrier stays all through the surgery, and until the time the skin suturing is done.

Description

The occlusive dressing (SSOD-1 FMS) is supplied in a pouch. Its superficial surface is called the ‘superficial surgical surface’ 1, and the deeper surface is called the ‘deep skin surface’ 9. The superficial skin surface 1 is the surface that forms the sterile surgical field to operate on, and the deep skin surface 9 will be in contact with the patient's skin, forming an impervious barrier, that prevents contact between the patient's skin, and the rest of the sterile surgical field. The surgical dressing, being rectangular in shape, has two smaller edges 3, and two longer edges 4.

The Superficial Surgical Surface

See FIG.-1A, that shows the superficial surgical surface 1 of the dressing-1. (SSOD-1 FMS). It is the non adherent surface, and it is going to be the clean surgical surface of the occlusive dressing to give the incision on, and be draped as the surgical field. It has ‘sterile edge folds’ 2 on its smaller edges (of the rectangle) 3. They are about 3-4″ wide. It is just an extra layer covering the surface of the occlusive dressing at these edges. They are closed at the outer edges 3 , but open towards the central or the operating side 5 of the dressing. In other words it is similar to the sleeve end of a shirt, or apron, folded up. In its closed edge, it has small openings at few places 6, so that fluids (ex-like amniotic fluid), would not collect there during surgery. Inside the folds, at both the corners and the center, it has small loops 7 that help easy handling of the dressing by the surgical team, with out touching the outer edges that are touching the patient's skin. The other functions of the ‘sterile edge folds’ will be described soon. The sterile surgical occlusive dressing has rabbit ear like loops 8, bigger than the loops described for the sterile edge folds, at all corners out side, to facilitate easy handling of the dressing.

The Deeper Skin Surface

See FIG.-1B. It is the adhesive deeper skin surface 9 of the (SSOD-1 FMS), that will be attached to the patient's skin, and is covered by a detachable adherent sheet, the model of which can be compared to a wall paper cover. The covering sheet is not a single continuous piece, but rather has 3 separate sheets for easy detachment, while securing the dressing to the patient's skin. The central piece 10 is few inches bigger than the two side pieces 11. Their line of separation is marked by a colored lines on the surface, or 1″ width overlap of non adherent appendages 12 on either side of the central piece on to the two adjacent ones. The dressing has to be attached to the surgical site with out touching the patient's skin.

The covering sheets of the adherent surface of the two smaller pieces also, are provided with a non adherent appendages 13, on their outer borders, 1″ wide, running the whole length of their edges They can be caught hold of, to detach the two covering smaller pieces from the deeper skin surface of the occlusive dressing.

The dressing is supplied in a pouch, and the pouch has written on the side of the deeper skin surface 9, as ‘ADHESIVE’, and on the side of the superficial surgical surface 1, as ‘NON ADHESIVE’, and can also be colored differently, for easy identification of the surfaces.

How to Use the Occlusive Dressing-1 for Major Surgeries (SSOD-1 FMS)

If there is a concern of the skin landmarks not being visible or clear, they can be marked before by a bright colored marker, and they can be visible through the transparent occlusive dressing. Or small ½ cm. diameter dome shaped buttons or cylinders can be taped to the skin over any landmark or desired point to be obvious through the dressing. However the team has to make sure that they do not slip into the subcutaneous thickness of fat when the dressing is removed and it is especially possible in very obese abdomen, and so these buttons or cylinders can have long thread trackers with their ends to stay out side the surgical field with a hemostat. After the patient's skin preparation is done, the dressing-1 (SSOD-1 FMS) has to be removed from the pouch. Before doing that, the deeper skin surface 9 has to be identified, and it has to be facing upwards, while being spread onto the instrument table. First the central piece 10, of the covering sheet of the deeper adhesive skin surface has to be removed, by the assisting nurse holding the appendages 12, and the dressing handed over to the operating team, with the nurse holding the rabbit ear loops 8 on the length wise side 4 of the dressing, which would expose the superficial surface of the dressing also. The assisting surgeon can pass both the hands into the sterile end folds 2 holding it with the flat of his hands (hands out stretched, fingers unfolded), and spreads the dressing, just above the abdominal surface, stretching his arms outwardly. Depending upon where the incision is, making it as the central point, the surgeon guides and secures the exposed adherent central piece of the dressing on to the incision skin site first, and then proceed lateral wards with both hands, thus attaching the central piece first. This facilitates a smooth attachment without folds in the incision site. Now the surgeon can catch hold of the appendage fold 13, of the small covering sheet 11, on one side with a hemostat or hand, and the sheet can be detached. Now starting from the central area and proceeding towards the periphery, it can be attached to the patient's skin, with out creating too many folds. The same can be repeated on the other side. After that the hemostat can be discarded. Some other person, not scrubbed, can also detach the small covering sheets by catching hold of the extra appendage at the outer border, while the surgeon tries to secure it to the patient's skin starting near the incision side, making sure not to create too many folds in this area, and proceeding towards the periphery. The same procedure can be repeated on the other side also, which completes the attachment of the dressing to the patient's skin. The whole time taken for the attachment to the skin would be less than 5 minutes, and it is done with out the surgical team touching the patient's skin all through. The size of the dressing has to be selected in such a way, that it securely covers a reasonable area of the skin, in the vicinity of the incision.

Now the abdomen can be draped with sterile towels in the usual manner and the incision can be given on the skin surface covered with the dressing (SSOD-1 FMS). The skin towels can also be secured to the skin edges with the towel clips in the usual way. The sterile surgical dressing-1 stays all through the surgery, and until the skin suturing is done, which can be either by mattress suturing, or by stapling. At this point the surgical dressing-1 has to be removed. The surgeon and the assistant can pass their hands into the sterile end folds 2, catch hold of the small loops 7, in the corners and in the center, and can lift the occlusive dressing from the patient's skin. Because of the end folds 2, this removal of the surgical dressing can be done without touching the patient's skin. If additional help is needed, the rabbit ear folds 8 at the corners of the dressing can be caught hold of with a sterile hemostat by the surgeon, or by some body from the non surgical team to facilitate the removal of the surgical dressing. Now the skin is ready for skin suturing, which is also done in a sterile fashion, with the help of surgical occlusive dressing-2 for major surgeries, secured to the surgical site, as going to be described in the following page.

Sterile Surgical Occlusive Dressing-2 for Major Surgeries (SSOD-2 FMS)

Description

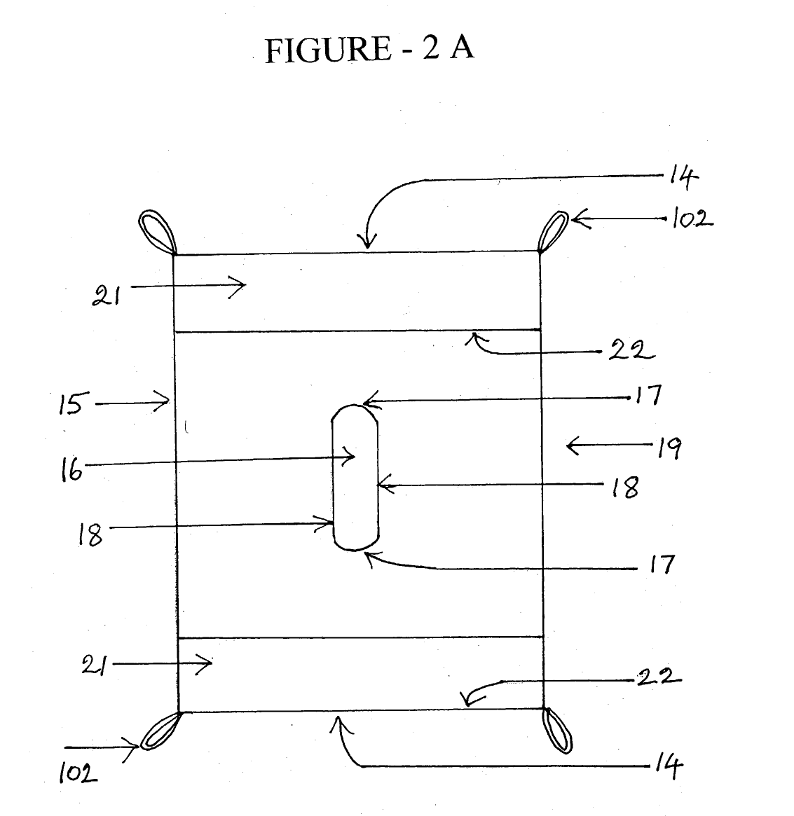

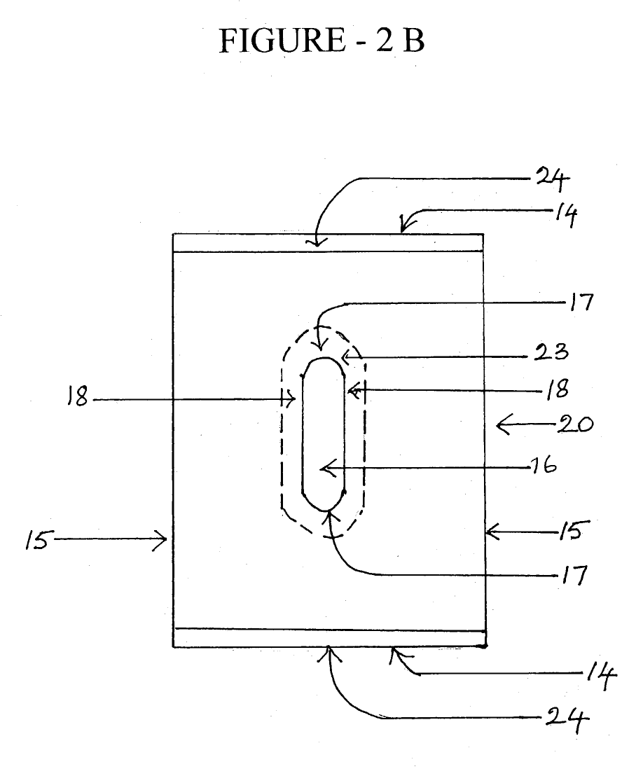

The dressing-2 (SSOD-2 FMS) is also enclosed in a pouch, which is opened at the time of suturing of the skin. It is also made up of water proof, transparent polyurethane, and the measurements can be chosen as the same, or can be lesser than the dressing-1 (SSOD-1 FMS) for any given patient. It also comes in different sizes with 2″ difference, varying 15-25″ width and 20-30″ length wise, and is mostly rectangular with smaller sides 14 (the width), and the longer sides 15 ( the length). It has a long central elliptical opening 16, as shown in the FIGS. 2A and B, with two corners 17, and two long edges 18. The width of the opening 16 is about 4″, and it's length ranges from 4-12″, the dressings being available with a difference of 1″ length, to suite different sized incisions. It allows space for mattress suturing or stapling. If the length has to be extended certain times, it is a good idea to cut open at one end, 17, instead of both the ends. If the cutting is done after it is attached to the skin, the instruments used for cutting needs to be discarded.

It has rabbit ear loops 102 on all four corners. It also has superficial surgical surface 19 and a deeper skin surface 20.

The Superficial Surgical Surface

See FIG. 2A, that shows the superficial surgical surface 19. It also has ‘sterile end folds’ 21, similar to the SSOD-1 FMS, to facilitate easy handling while securing it to the surgical site. As described before, it is about 3-4″ wide. It is just an extra layer covering the surface of the occlusive dressing at the ends, closed at the edges 14 , but open towards the central or the operating side 22 of the dressing, and is similar to the sleeve of a shirt that is folded.

Deep Skin Surface

See FIG. 2B. The deeper skin surface 20 of the surgical occlusive dressing-2 is different from the dressing-1. The occlusive dressing-2 has 2″ wide adhesive margin 23, that makes the elliptical opening 16, on the deeper skin surface, and it also has adhesive edges 24 of 1-2″ wide, at its smaller ends 14 (of the rectangle), covered with a detachable covering, with it's non adherent appendage like ends projecting slightly outside the dressing, for easy removal. How ever, the other side i.e. the longer ends 15, and the rest of the deeper skin surface 20 are not adherent. This is to allow aeration, and not to allow too much perspiration postoperatively, because it stays on the patient's skin for 1 or 2 days after the surgery.

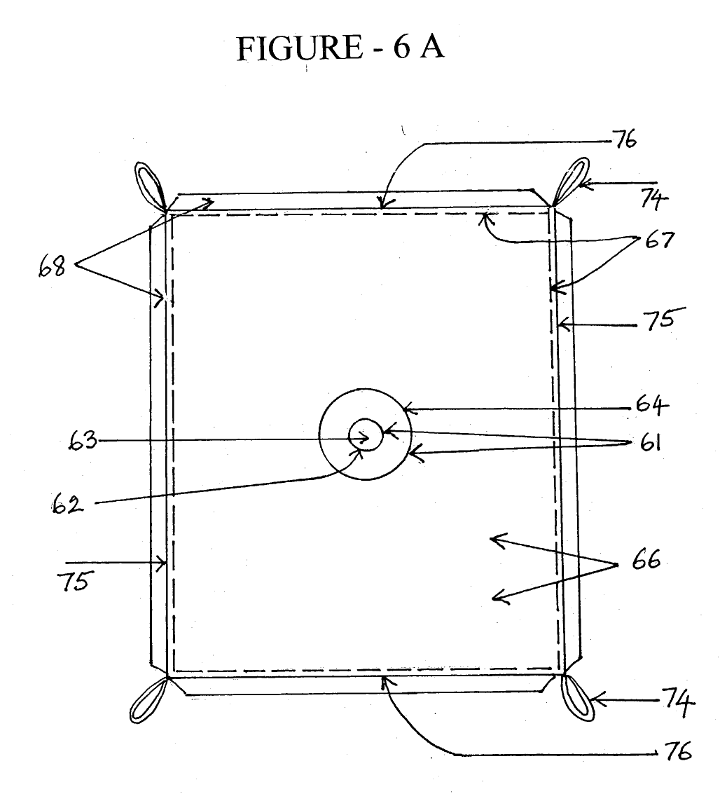

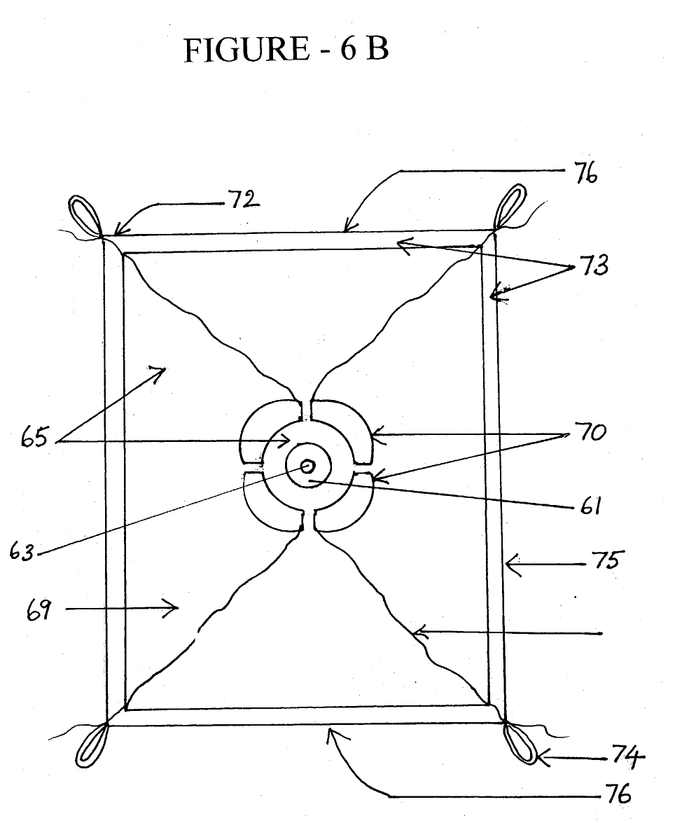

(With all the precautions taken so far, it is important to do the skin suturing also with antiseptic technique, because there is significant contact of the patient's skin with the needle and the suturing material, that are carried into the deeper planes of the skin and the subcutaneous tissues, thus causing both the superficial and deep incision site infections. So the incision edges of the exposed skin surface in the elliptical opening 16 is covered with the ‘sterile skin edge patches’, supplied separately (device 6, FIGS. 6A and 6B), on which suturing is done.

How to Apply

The assisting nurse, after removing the surgical occlusive dressing-2 from the containing pouch, spreads it on the instrument table, and removes the covering of the 2″ adhesive surface 23 of the elliptical edges of the deeper skin surface. Then, she hands it over to the surgical team holding it in such a way, that the superficial surgical surface 19 is facing upwards. The assistant can pass his hands into the sterile end folds 21, thus holing it with the flat of the palms, and spreading his arms outwardly, but not catching hold of any part of the surgical dressing in particular. He can hold it like that, just above the surgical site, while the surgeon can direct it to the exact site, so that the elliptical opening 16 is precisely around the incision. Then the surgeon can secure the adhesive edges 23 of the elliptical opening 16 to the patient's skin, around the incision, with out touching the patient's skin. Now the assistant can rest the whole dressing on the surgical field except the edges he is holding. Some body, who is not scrubbed, or the surgeon, can remove the covering of the adhesive edges 24 of the smaller ends 14, as they are still above the skin, so that they can also be attached to the patient's skin, but not all the way up to the edges. Now the area is draped, and at this point of surgery, it does not necessarily be the cotton fabric towel draping.

Alternatively, the attachment of the edges 24 can be done at the end of the suturing, or just before the patient leaves the operating room, which should not be forgotten, because the dressing is holding itself to the patient's skin only by the elliptical adhesive area, but the surgical team doing it, gives the added protection of avoiding unnecessary contamination of the vicinity of the patient's skin, post operatively, by the patient or the other personnel of the operating room.

Sterile Surgical Occlusive Dressing-1 and 2 for Minor Surgeries

Description

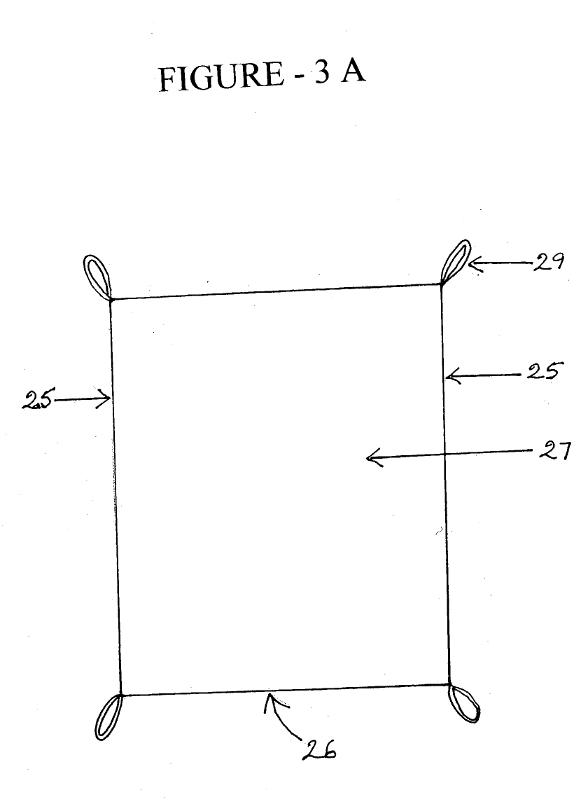

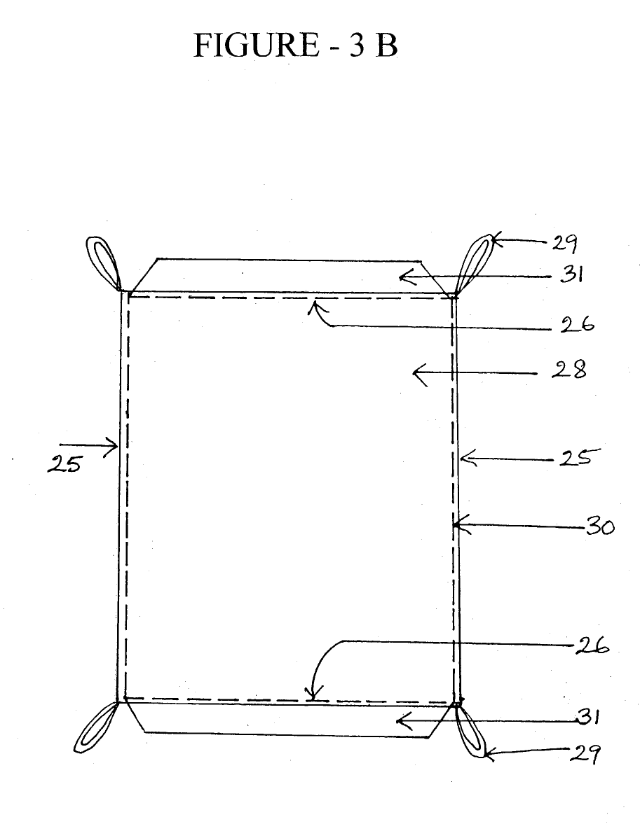

For minor surgeries (referred so, only due to the small incision involved), or the surgeries that do not need a very big incision, like head and neck surgeries, or the ones on the extremities, the surgical occlusive dressing needed, can be small, measuring 5-10″×5-15″, with size ranges each of 1″ difference. See FIGS. 3A and 3B. These are also made up of thin, water resistant polyurethane, transparent dressing, and also has superficial surgical surface and a deep skin surface.

Sterile Surgical Occlusive Dressing-1 for Minor Surgeries (SSOD-1 Mini Surge)

The occlusive dressings 1 for minor surgeries, has two longer sides 25 (the length) and two smaller sides 26 (the width).

It has superficial surgical surface 27 (FIG.-3A) that forms the surgical field, and a deep skin surface 28 (FIG.-3B), that will be secured to the patient's skin.

The dressing has the rabbit ear loops 29, in it's corners for easy handling.

The Superficial Surgical Surface

See FIG.-3A. The superficial surgical surface 27 of SSOD-1 mini surge., after the dressing is secured to the patient's skin, forms the surgical field to operate on, but it does not have the end folds, because it can be easily secured to the patient's skin even with out them.

The Deep Skin Surface

The deep skin surface 28 of the SSOD-1 mini surge as shown in the FIG.-3B, has only one piece of adhesive covering 30, that has small appendage like non adherent out ward projections 31 on the smaller side 26 of the dressing for the easy handling of the covering 30 during removal.

The Surgical Occlusive Dressing-2 for Minor Surgeries (SSOD-2 Mini Surge)

The surgical occlusive dressing-2, for minor surgeries (SSOD-2 mini surge), also has a superficial surgical surface 32, (FIG.-4A), and the deep skin surface 33 (FIG.-4B), two longer ends 34 (the length), and the smaller ends 35 (the width), with a size range of 5-10″×5-15″, with size range each of 1″ difference, and has an elliptical opening 36, in the center of the dressing. The elliptical opening has two curved ends 37 and has a width of 2″, and two margins 39, both with a length of the range 3-8″. It also has the rabbit ear loops 40, in the corners, for easy handling of the dressing.

The Superficial Surgical Surface

After attaching the dressing to the patient's skin , the superficial surgical surface 32, as shown in the FIG.-4A, forms the surgical surface to operate on. Unlike the major surgical dressing, it does not have the end folds, because it can be easily handled, even with out them.

The Deep Skin Surface

The deep skin surface 33, as shown in the FIG.-4B, is the surface of the dressing that is secured to the patient's skin. It is similar to the bigger dressings in having adhesive edge 41, 1″ wide, to the elliptical opening 36, the adhesive surface having a detachable cover. In it's smaller ends 35 (the width), it also has adhesive margins that have detachable covers 42, with small appendage like non adherent projected ends 43, out side the edges for easy handling.

If the elliptical opening has bigger length than desired, it should not cause concern, because the extra length can always be covered by the sterile skin edge patches, if the incision is very small.

How to Apply the Dressings 1 and 2

After the skin is scrubbed, the SSOD-1 mini surge, is taken out of the sterile container it is supplied in. The nurse can spread it on the instrument table and after removing the whole adhesive covering 30 of the deep skin surface, hands it over to the operating team, holding the 2 rabbit loops 29, in the corners. The surgical team can catch hold of the other two loops 29 while receiving, and after that all the four loops can be held on by two people of the operating team, to conveniently apply on to the skin with out causing too many folds in the center where the incision is given. Or the assistant can hold the center of the two ends 26, just above the skin, while the surgeon can secure it to the skin at the incision area first, and then proceed to either side. Going all the way up to the skin is avoided at this point, so that the patient's skin is not touched. Now the surgical field can be draped in the usual manner, and at this point the edges of the dressing can also be attached to the skin, by pressing over the draping that are applied over it. Now the incision can be given on the superficial surgical surface, and the skin towels clipped in the usual manner. It stays all through the surgery, and until the skin suturing is done, when it can be removed by catching hold of the rabbit ear loops 29 at the corners with the hemostat, with out touching the skin.

Now, the SSOD-2 mini surge, has to be applied to the skin. The nurse has to remove the cover on the adhesive edge 41, and holding the rabbit ears 29, hand it over to the surgical team. The assistant can hold the center of the smaller edges 35 on either side, while the surgeon guides it to the incision site, to be attached to the center first, then proceeding to the edges, all the while not touching the skin in the center or the periphery. Now the surgeon can remove the adhesive coverings 42 of the edges 35, one at a time, as they are still above the skin surface, so that they can be secured to the surgical surface, but not all the way up to the skin, to avoid touching the skin. Now the skin can be draped with the towels, and the edges of the dressing can now be firmly secured to the patient's skin. It does not necessarily be the cotton fabric towels to use at this point of surgery.

As described before, this surgical occlusive dressing - 2 for minor surgeries also works in conjunction with the sterile skin edge patches, to cover the elliptical space, and the incision margins, before doing the skin suturing.

Sterile Surgical Occlusive Dressing for Urological Surgeries

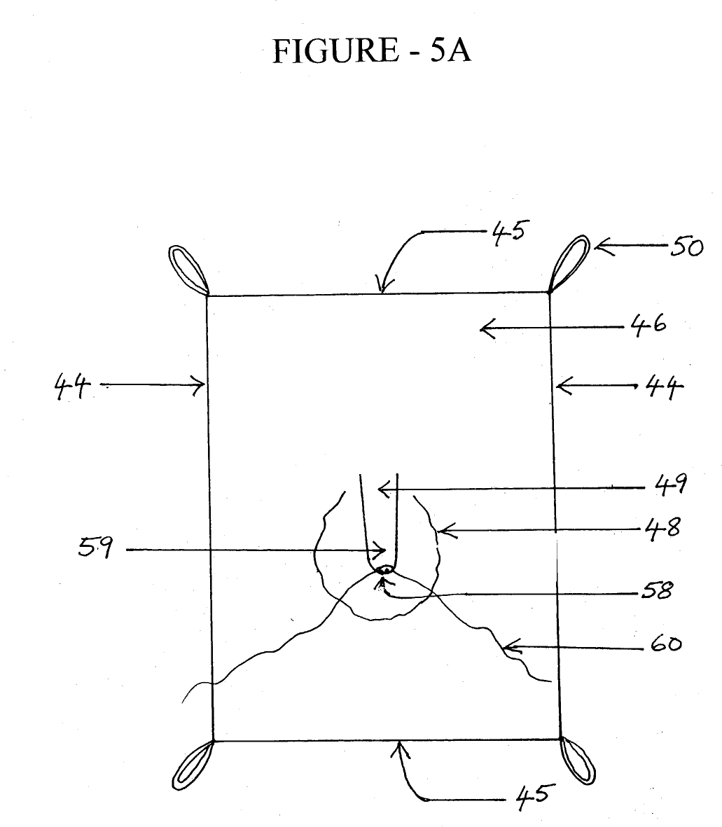

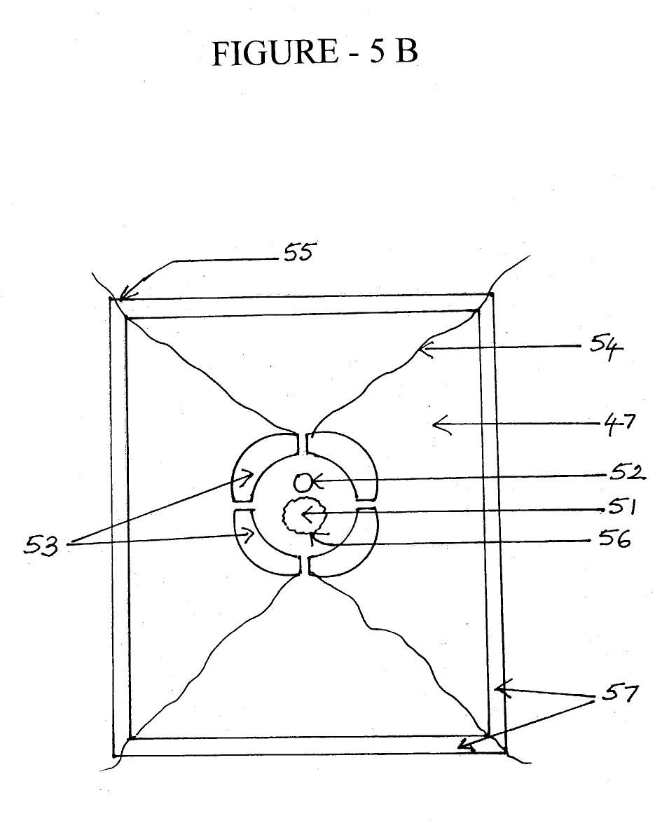

Transurethral urological surgeries need special mention, as they need special type of sterile surgical occlusive device. The anal, perineal and scrotal skin can never be satisfactorily cleaned, and surgical contamination through these areas, especially the anal area, is hard to avoid even in the most meticulous hands. Though these areas are covered by sterile drapes, slipping of the drapes, accidental touching during difficult manipulations, can happen very easily. A specially devised dressing shown in the FIGS. 5A and B, can be used during these surgeries to help minimize contamination.

Description

The surgical occlusive dressing for urological surgeries is of size range of 20-25″×35-40″, with 2″ difference in the size ranges, and is made up of thin water resistant, polyurethane. It has two longer sides 44 (the length), and two smaller sides 45 (the width), a superficial surgical surface 46, and a deep skin surface 47. The dressing has a scrotal pouch 48 in the center, and just above it, a penile cover 49. It is also supplied in a plastic container. It has rabbit ear like loops 50 at the corners for easy handling.

The Deep Skin Surface

See the FIG.-5B. The deep skin surface 47 is the surface attached to the patient's skin and has a ring like incomplete circular adherent area, about 2″ wide, located 1″ from the opening 51 of the scrotal pouch, and the opening 52 of the penile cover. The circular adherent area is divided into 4 quadrants of a ring covered by detachable pieces 53. A thin string 54 is attached to each of these 4 pieces, whose other ends are anchored to the corresponding corner 55 of the dressing. The scrotal pouch has an elastic border 56 for appropriate fit. The deep skin surface of the dressing also has adhesive borders with cover 57, 2″ wide all around it's borders, that has 1 cm. non adherent appendage projecting out, for easy handling.

The Superficial Surgical Surface

The superficial surgical surface 46, shown in FIG.-5A, becomes the surgical field to be operated on, while spread on the surgical field with the patient in the lithotomy position. It is covered with a thin plastic sheet, only attached at the margins of the dressing, when taken out of the pouch it is supplied in. This sheet is removed by some body out side the surgical team after it is spread on the surgical site, holding the rabbit ear loops 50 to do that, starting at the caudal side of the patient and proceeding upwards, all the while not touching the surgical surface that is being exposed. Doing this way enables the surgeon also participate in this process, as he adjusts the dressing with his hands on the surgical surface, which makes it easier when it is being done at the caudal end. After the covering sheet is removed, the scrotal pouch and the penile cover are seen on the superficial surgical surface of the dressing.

The penile cover 49 is made up of tight, but very expandable condom like rubber that has a very small opening 58 at the tip 59, which gets dilated when urethral instrumentation is done. It has two threads 60, 9-12″ length, on its either sides, for the identification of the hole. Stretching the two threads laterally can identify the hole during the time the initial instrumentation is done ( the threads can be clamped to the thighs ). The edge of the hole is thick and tire like, and at the end of the surgery, the surgeon has to identify that the entire margin is intact, and there is no broken piece stuck in the urethra, that can cause infection, at a later date.

How to Use It

After cleaning the patient's skin the surgical occlusive dressing can be removed from the container pouch, and handed over to the nurse, who spreads it on to the operating instrument table to identify the superficial surgical surface 46 and the deep skin surface 47. The superficial skin surface is covered all over with the transparent plastic sheet, and the deep surgical surface has the circular adherent surface 53 and also the adherent edges 57. Then she hands it over to the surgeon who is standing facing the patient in the lithotomy position. The surgeon, spreads it on the surgical area. Some body not scrubbed has to remove the covering sheet of the superficial surgical surface 46 starting at the edge of the caudal end and proceeding upwards, not touching the surgical surface. The surgeon also helps in the process without contaminating his hands. Now, holding the surgical dressing on the surgical surface 46, he has to insert the penis into the penile cover 49 first, and then spread the scrotal pouch 48 around the scrotum, with both the hands. The base or the opening 52 of the penile cover 49, is elastic, which facilitates easy insertion of the penis into the penile cover. At this point, some body who does not belong to the operating team, can pull the strings 54, of the covers 53 of the circular adherent surface, one at a time while the surgeon simultaneously tries to attach it to the patient's skin. After all the four quadrants are attached, the dressing can be adjusted to the contours of the patient's body in the lithotomy position. Now the same assistant, holding the edges of the dressing, can remove the cover 57 of the adhesive edges on all the four sides, and also secures the edges to the patient's skin all around, while the surgeon can also help with his hands on the surgical surface of the dressing, without touching the patient's skin. There can be lot of folds because the patient is in the lithotomy position, but that would not pose a problem, as long as it stays in position, and covers the patient's skin. The patient can be draped with the sterile towels over the dressing, in the usual manner, before proceeding to the surgery.

Sterile Surgical Occlusive Dressing for Obstetrics and Gynecological (OB/GYN) Surgeries.

Description and Use

See FIGS.-6A & B that shows the sterile occlusive dressing for vaginal ob./gyn. surgeries, a polyurethane, water resistant rectangular 20-25×35-40″ size range dressing, with 2″ difference in each available size, can be used to cover most of the external genitalia around the introitus, the thighs, and the lower abdomen. The dressing in the center, is made up of very expandable rubber ring 61, that has two concentric circular edges, the inner one 62 about 1″ in diameter, that encircles a central hole 63 and the outer 64 about 2″, to which the rest of the sterile plastic occlusive dressing sheet 65 is attached making this edge look like the elastic cuff of a sleeve. The inner edge 62 of the rubber ring can be sutured into the vaginal wall at few (4) places, few millimeters from the junction of the vaginal and vulval skin. It can be conveniently done by stretching the vaginal opening, along with the rubber ring 61 of the dressing, using a Sim's speculum, and turning it around the vaginal wall to facilitate the ‘attachment suturing’. The top and the bottom suturing can be done first, then the two lateral suturings, or vice versa. This way of doing makes the four sutures equidistant with out any uneven pull on any quadrant. Additional sutures can also be done if the surgeon desires. The rubber ring 61 is rather thin and flat, instead of thick or tire like, and it can be thinner at its inner most edge to facilitate easy suturing to the vaginal wall.

The surgical dressing also has superficial surgical surface and deep skin surface. The superficial surgical surface 66 as shown in FIG.-6A, is covered by a transparent plastic sheath that is attached to the surgical surface 66 only at it's margins 67 with 1 cm. non adherent edge 68 projecting outside the dressing for easy handling and removal.

The deep skin surface 69 as shown in the FIG.-6B, has an incomplete circular adherent area of 2″ width, located 2-3″ from the outer margin 64 of the circular rubber ring, to be attached to the skin adjacent to the external genitalia. The incomplete circular ring shaped covering of the adherent area located 2-3″ from the circular rubber ring 61 on the deep skin surface 69 is divided into 4 separate pieces 70, one located in each quadrant of the circle. Each piece has a thin string 71 attached, which is anchored to the corresponding corner 72, of the surgical dressing. The deep skin surface 69 also has adherent margins 73, about 2″ wide in all four sides covered by adherent cover with 1 cm. appendages, projecting out, for easy handling. The dressing also has the rabbit ears like loops 74 in the corners.

Just before the surgery, some body from outside the surgical team hands over the dressing to the assisting nurse, taking it out from the pouch it is supplied in. The nurse can spread it on the instrument table, and identifies the superficial surgical and the deep skin surfaces. The superficial surgical surface 66 is covered by the transparent plastic sheath on it's whole surface, and the deep skin surface 69 has the circular ring shaped covering 70 and also the covering at the edges 73. The nurse can hand it over to the surgical team, and they can spread it over the operating field, with out touching the patient's skin. Some body from out side the surgical team can help remove the plastic covering over the superficial surgical surface 66 starting at the corners of the caudal end, holding the rabbit ear loops 74, but not touching the surgical surface 66 as it is being exposed. The surgical team can also help the process, with out touching the patient's skin. Now the surgeon can identify the central hole 63 of the rubber ring 61 and maneuver it to the vaginal opening, and can introduce the Sim's speculum through the opening 63 into the vagina. As the speculum is covering the posterior vaginal wall, the ‘attachment suturing’ of the inner edge 62 of the rubber ring 61 can be started in the anterior vaginal wall. After that, the speculum can be turned to the anterior vaginal wall, so that the attachment suturing can be done in the posterior vaginal wall, and so also the two lateral stitches. After the vaginal attachment suturing is done, an assistant who is not participating in the surgery, can help in removing the coverings of the circular adhesive surfaces 70, from the deep skin surface 69 of the dressing, by pulling on the string 71, attached to the corners 72 , while the surgeon tries simultaneously to secure that part of the dressing on to the patient's skin. This attachment prevents too much pull on the vaginal ‘attachment suturing’, if the surgery is very prolonged, and it can be avoided also for short surgeries. After this, the adherent edges 73 can also be attached to the patient's skin, and it can be done by some body out side the surgical team. The surgical field can be draped with the sterile towels in the usual manner after the dressing is secured to the patient's skin.

The length of the dressing 75 can be used to cover the length of the patient i.e. the vertical aspect of the abdomen, and the vertical aspect of the thighs, where as the width 76 of the dressing is used to cover horizontally the abdomen and the thighs.

During the completion of the surgery, as the vaginal suturing is approaching the introitus, the ‘attachment suture’ can be cut to complete the suturing. After the surgery, the occlusive dressing can be taken off, and the patient does not need the one like surgical occlusive dressing-2, because the surgical site deep in the vagina is isolated from the surface skin.

It is a good idea to pack the vagina with ribbon gauze soaked with the antiseptic of choice, the night before the surgery to keep over night and to remove it in the operating room just before surgery. It cleans most of the vaginal mucosa, the ectocervix, and to some extent the very distal part of the endocervix. However the vaginal mucosa, more accurately called the vaginal skin, is still a vulnerable area that is not covered during surgery. Still what is hoped is, to protect the surgical field from the overt contamination from the anal and perineal areas, by covering the area out side the vagina in the best possible way, by the sterile surgical occlusive dressing as described.

The Sterile Skin Edge Patches

After all the antiseptic precautions taken to avoid the contamination with the patient's skin until the time of the skin suturing, it is imperative that the skin suturing is also done with the same antiseptic principles. There is significant contact of the needle and the suturing material with the patient's skin, and they are carrying the skin bacteria into the superficial and deep subcutaneous planes, when the mattress suturing is done. The situation is similar, but to a lesser extent, when stapling is done as the mode of skin closure. For this reason, the ‘sterile skin edge patches’ are used in conjunction with the surgical occlusive dressing-2, to make the suturing done with antiseptic precautions. Before the surgical dressing-2 is applied the skin has to be cleaned thoroughly of blood or other body fluids, so that the skin patches can be applied over cleaned skin.

Description and Use

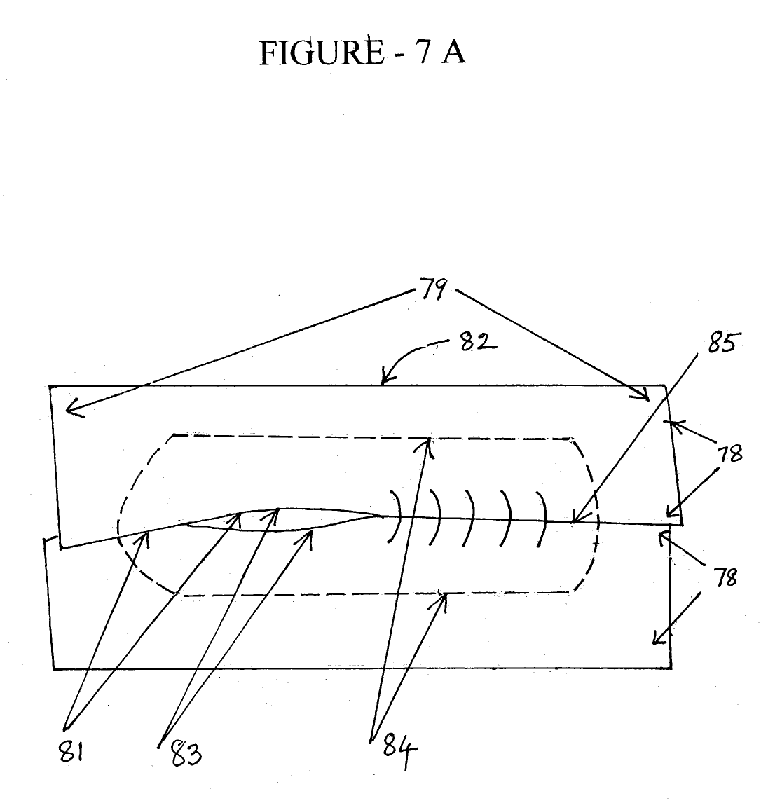

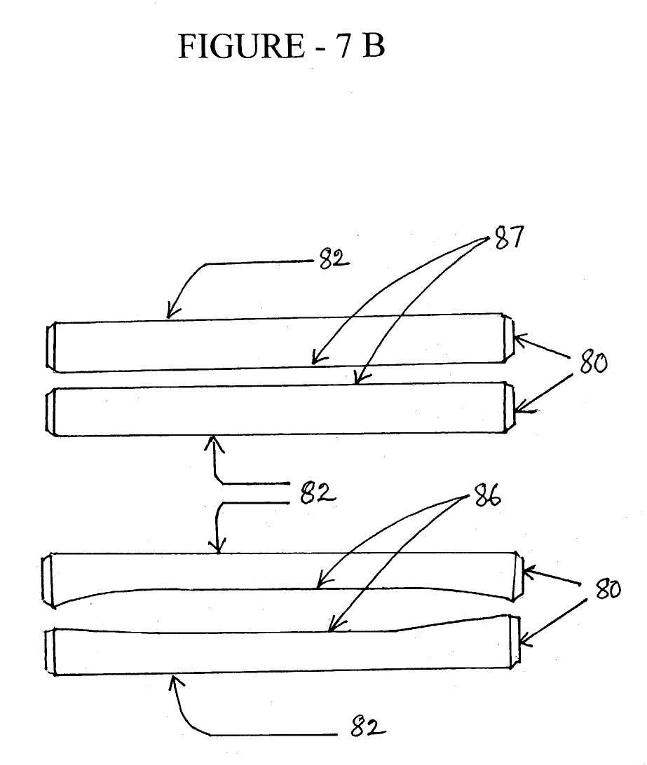

See FIGS. 7A and B. The sterile paper skin edge patches 77 are made up of sterile paper patches that have a width 78 of 3-4″ in size, and can be made of different standard lengths 79 to suit different lengths of the incision. They can be made of with 1″ difference and can be numbered as per their lengths in inches i. e. a 10″ patch can be numbered 10. They have a covered adhesive deep skin surface and a non adherent superficial surgical surface. The covering sheet of the adherent surface has 1 cm. appendage like projection 80 on either side of it's length (see FIG.-7B), for easy removal of the covering of the adherent surface when it is being attached to the incision area. the patch has a medial margin 81 and a lateral margin 82. The patches can be soft but moderately tough, and comparable to paper towels in thickness and pliability. See FIG. 7A. These paper patches can be secured to the incision margins 83, and can completely cover the incision's skin margin 83, and the edges of the elliptical opening 84 of the surgical occlusive dressing-2, on all the sides including the corners so that there is no exposed skin at all. So the length of the skin edge patches 77, selected should be 3-4″ longer than the incision itself, so that the extra length covers the skin adjacent to the angular corners 85 of the incision, and also goes over the edge of the elliptical opening 84 of the dressing so that there is no exposed skin in between. If there is a problem in approximation of the patches in the median plane, in the area adjacent to the corners 85 of the incision, another small piece of the patch can be applied over the separated ends of the patches to cover the skin. The idea is only to cover the skin, and so can be done in any way that is convenient. It is not a problem, if there are some folds, or if it is not aesthetically appealing. How ever, in practicality, there should not be any difficulty because it is made up of paper, and is pliable; so when the corner angles 85 are being covered, even if there is significant separation, the patches can be sufficiently bent medially for approximation to cover the skin, though causing some folds. It is also a good idea to select a patch 1-2″ longer than desired, cut that extra length to be used for any uncovered areas of the skin.

However, for a very obese abdominal wall, even if the fat and subcutaneous tissues are meticulously sutured, there can be significant gaping in the incision to be sutured, in which case a slightly curvilinear edge of the medial edge 81 of the patch with medial concavity 86 (See FIG. 7B), rather than a straight 87 of the medial edge 81 of the patches can be used, which can completely cover the edges 83 of the incision. All this has to be done without touching the patient's skin. It is a good idea to first attach it at the center of the incision by the surgeon, and then proceed on either side, while the assistant is holding it at the ends. Even if there are some folds, it does not make a difference in the way the mattress suturing is done. It is also advisable to go beyond the skin edge, rather than leaving some skin uncovered over the edge while manipulating the attachment. These edges of the patch that projects beyond the skin can be caught hold of with a hemostat, while the mattress suturing is tightened, to avoid it being tucked in between the sutured incision while it is tightened. If still there are some thin exposed margins of the incision skin, taking skin bite there can be avoided. The procedure of attachment should not be hard, because the patch completely covers the exposed skin area and as there is sterile area all around, it makes the manipulation with the hands easier.

The pair of skin edge patches are supplied in a pouch with their length mentioned on the cover. So it is a good idea to have a sterile measuring tape on the operating table, to know the exact size of the incision. How ever with experience, the surgeon can know the size of the incision, with out the help of the measuring tape. The type of the patch, either straight or curvilinear, is also mentioned outside the pouch. If the surgeon starts with a straight patch but has difficulty approximating, the patch can be dislodged from it's attachment by the assistant lifting at it's edges, and the surgeon can create few folds on the medial margin of the patch, through it's superficial surgical surface which can create a curvilinear shape that can suit the contours of the incision.

After the suturing is complete, the paper patch 77 can be left over and an antibiotic ointment in excess applied over the patch, and a dressing can be done. Usually there will not be any signs of infection until the 3rd or 4th post operative day. After that, if there is any suspicion of infection the patch can be soaked with betadine or hydrogen peroxide for few minutes, and the paper scrapes or crumbles can be removed with the fingers or with the help of a forceps to inspect the incision area. Even if there are some leftovers, they would not come in the way of inspecting the sutures post operatively, for any signs of infection, swelling, induration, or drainage. At this time sufficient scab would form over the incision so that the skin bacteria would not gain entrance to the subcutaneous planes, the danger of which is possible if the patch is removed immediately after the surgery, by soaking it, when the skin bacteria can force their way into the incision to enter the deeper planes.

Sterile Transparent Plastic Water Tanks

Hand washing with sterile water after 5 minutes of scrubbing, is an ideal way to minimize avoidable contamination. It is very hard to sterilize the water source of the operating room i. e. the water tanks, the piping system and the water faucets as per the surgically satisfactory ways. So an easy and a practical way to do it, is to use 10-15 gallon size transparent, sterile, disposable (but recyclable), plastic water tanks for hand cleaning. These tanks or cylinders can be placed in a frame fixed to the wall above the sink of the washing and scrubbing area. Each sink can have a set like this.

Description and Use

See FIGS.-7A, B and C. Each water tank 88 is made up of 10-15 gallon size transparent, sterile, disposable plastic water container. The water tank or the cylinder has a stopcock 89 above its faucet area 90 and when it is supplied it is sealed and closed with a cap 91, does not have the attached water tap, but how ever has a provision for the attachment, after the cap 91 is removed. The Sterile plastic water tap 92 in a plastic pouch 93 (FIG. 7B) are separately supplied (recyclable), along with the water tanks 88. It saves space if the tanks stay horizontally in the frame above the sink, instead of standing vertically. Just before use, the scrubbing personnel should close the stopcock 89, remove the cap 91 and the seal, and replace it with the plastic tap faucet 92. To do that, the transparent pouch 93 of the tap faucet has to be opened on the side 94, on which side the tap faucet has the opening 95 that fits to the water tank. After fixing it (see FIG. 7C) the pouch 93 can be removed. All this is done while the tap faucet is still in the pouch 93, and not touching the water faucet with the hands. During this process, make sure that the faucet is in the ‘off’ position, the way it is supplied in the pouch, but can be disturbed during the time it is fitted to the tank. Now the surgeon can turn on the stop cock 89 , but not the tap faucet 92 , before starting to scrub. After scrubbing, the plastic water faucet 92 can be turned on by it's ‘on and off’ stop cock 95, by the person who is scrubbing with out any body's help, to wash the hands with the sterile water. After that, the surgeon can also close the tap himself with out some body's help, and with out wasting water. When the next person comes to wash, he/she can close the stop cock 89, remove the used plastic disposable water tap 92, and fix a new one, like the way described before. After that he/she can open the stop cock 89 and start the hand scrubbing. If the surgeon thinks that the water in the tank is not sufficient for the entire scrubbing, he can fix another fresh disposable tap in the adjacent sink area also before starting to scrub, or he can let some body help him if he runs short of water unexpectedly. The water tanks can be made available in different weights, so that the operating personnel don't need to wait for some body in situations of emergency, if the bigger water tank is too much to carry. How ever, the working people of the operating area, while moving the patients in and out of the operating room, should be always alert to refill them immediately, and before leaving the operating room at the end of the week days, and at the end of each surgery during week ends and after hours, so that water is immediately available for emergency surgeries, when they are rushed into the operating rooms. The frames can also be made in such a way to reach the roof, so that 3-4 water tanks can be placed at one time, with a provision to slide and refill when the bottom one is used. If the sliding arrangement can go up and down, it is easy to refill the bottom space than the top ones. These water tanks are to be used only for surgical scrubbing, and the regular tap water in the sinks below, has to be used for the rest of the purposes, like cleaning the hands after the surgery . The plastic tap pouches 93 should be provided in a large container in the scrubbing area, near the sink. The water faucet 92 that is supplied in the pouches is a 2″ length, 1 cm. internal diameter, transparent, straight plastic pipe, with ‘on and off’ device 95. Its opening 96 that fits to the water cylinder, is broader with threading inside, or it can be colored differently, for easy identification. This type of straight structural configuration instead of the J or L shaped, suits for it to be fitted to the bottom of the cylinder, so that the flow of the water is to the center of the sink, which is not possible if it is fitted in front. How ever, because the tank is a cylinder, it only has to be rotated to bring the tap to the bottom of the cylinder. So, the bottom of the wall frame should have opening to accommodate the faucet.

The water tank 88 with the stop cock device, can be used for the irrigation of the surgical site also, by connecting the water tanks to sterile, disposable (recyclable) plastic water tubing 97, of moderate length, connected to the water tank by threading device 98 and it has an ‘on and off’ faucet device 99 at the irrigation site 100, to be used in the operating field. The tubing 97 with the faucet 99 is supplied in a plastic sheath 101 , which can be opened when needed by any body not scrubbed, and the faucet can be picked up by the operating people along with the length of the tubing needed in the surgical field as the sheath 101 can be simultaneously with drawn by the person who opened it, and the other end of the tubing with the threading device 98 can be connected to the water source. It is a good idea to keep the water source i.e. the water cylinder as near to the operating site as possible so that the tubing does not need to be very lengthy.

Fitting the frames of the water tanks to the wall above the sinks does not need any extra space, and can be done in a small or big and tightly packed operating rooms. It's cost is definitely less than the cost involved in treating the post operative sepsis, and its safety definitely improves the antiseptic parameters and the quality excellence of the surgical antisepsis measures.

1. The sterile surgical occlusive dressing-1 for major surgeries (SSOD-1 FMS), supplied in a pouch, comprised of a rectangular shaped, sterile, transparent, water resistant, polyurethane surgical dressing, measuring 15-25″×20-30″, having
1. Superficial surgical surface that is non adherent,
2. A deep skin surface that is adherent and,
3. Rabbit ear like loops at all its corners.

2. The SSOD-1 FMS, of claim 1, whose smaller sides of the rectangular shaped device, has ‘sterile end folds’ of 3-4″ size, on the superficial surgical surface, closed at its outer side, and open at its central or operating side, and the two folds also having small loops in the center, and in both the corners for easy handling.

3. The SSOD-1 FMS, in claim 1, whose deep skin surface is adherent and covered with 3 detachable pieces—a bigger central one and two smaller adjacent ones.

4. The ‘sterile end fold’ of the SSOD-1 FMS as of claim 2, have ½ cm. size holes in the closed outer sides.

5. The adherent skin surface of the claim 3, covered by 3 detachable pieces, have appendage pieces for easy handling during detachment, the central piece having two, one on either side, and the adjacent side pieces have one each, at their outer edges.

6. The SSOD-1 FMS, of claim 1, is of sizes 15-25″×20-30″ and of the available ranges of 2″ in difference.

7. The claims are for sterile surgical occlusive dressing-2 for major surgeries (SSOD-2 FMS), supplied in a pouch, made up of a sterile, transparent, water resistant, rectangular, polyurethane surgical dressing,
with measurements of 20-25″×35-40″ comprising of,
1. Superficial surgical surface that is non adherent,
2. Deep skin surface, that has adherent areas, and
3. Rabbit ear like loops at four corners.

8. The device in claim of 7, that has central elliptical opening, 4″ wide, and 4-12″ range in length.

9. The superficial surgical surface of the device in claim 7, made up of non adherent surface with ‘sterile end folds’, on the smaller sides of the rectangular device, the end folds closed at the outer ends and open at the inner ends, and also have small loops inside in its outer edges, in the center and in the outer corners.

10. The deep skin surface of the device of claim 7, that has adherent areas
2″ width, surrounding the elliptical central opening in the center,
2″ width, in the edges of the smaller sides of the device, the rest of the deeper surface being non adherent.

11. The claims are for sterile surgical occlusive dressing for urological surgeries comprising of rectangular 20-25″×35-40″ size, polyurethane, sterile, water resistant, occlusive surgical dressing, supplied in a pouch, comprising of
1. Non adherent superficial surgical surface,
2. Deep skin surface, with adherent areas,
3. A scrotal pouch and a penile cover, with their openings on the side of the deep skin surface,
4. Rabbit ear like tops on all the four corners.

12. The sterile superficial surgical surface of the dressing of claim 11, is covered by a thin transparent plastic sheet.

13. The deep skin surface of the device in claim 11, that has adherent areas, 2″ wide
1. located 1″ from the opening of the scrotal pouch and the penile cover, incomplete circular in shape, and covered by four separate adherent covers in four quadrants, that have strings, that are attached to the corresponding corners,
2. Around all the four edges (borders) of the rectangular shaped device.

14. The scrotal pouch of the claim 11, has elastic border in its opening, to prevent sliding of the dressing.

15. The penile cover of claim 11, made up of very elastic rubber like a condom, with a small hole in its tip, whose edge is round and tire like, with two threads about 9-12″ long, attached on either side of the opening.

16. The sterile surgical occlusive dressing for urological surgeries of claim 11, with sizes of 20-25″×25-40″, is available with a size range of 2″ in difference.

17. The claims are for sterile surgical occlusive dressing for obstetrics and gynecological surgeries, which is a rectangular, sterile,
water resistant, polyurethane dressing of 20-25″×35-40″ size, and enclosed in a pouch, comprising of,
1. Superficial surgical surface, that is non adherent,
2. Deep skin surface that has adherent areas,
3. A central rubber ring with a central hole,
4. Rabbit ear like loops on all its corners.

18. The device in the claim 17, whose deep skin surface has following adherent areas
1. An incompletely circular adherent area 2″ wide, located 2-3″ from the outer margin of the rubber ring, whose adherent covering is divided into four separate pieces with strings, located in four quadrants, with the ends of the strings attached to the corresponding corners,
2. An adherent area all around its borders, 2″ wide.

19. The device of claim 17, that has a central elastic ring of very expandable rubber, 2″ wide, the rubber ring being thin and flat, with its inner edge more thinner, with a central opening of 1″ diameter.

20. The device of claim 17, with the sizes of 20-25″×35-40″, is available with the size ranges of 2″ in difference.

21. Sterile surgical occlusive dressing-1 for minor surgeries, made up of sterile transparent, water resistant, rectangular, polyurethane dressing about 5-10″×5-15″ size (with size range difference of 1″), comprising of,
1. Non adherent, superficial surgical surface,
2. Deep skin surface with adherent areas,
3. Rabbit ear like loops.

22. The deeper skin surface of the sterile surgical occlusive dressing-1 for minor surgeries, of claim 21, is covered by a single sheet, and adherent through out, with non adherent appendage like 1 cm. projections on the smaller sides, for easy removal.

23. The sterile surgical occlusive dressing-2, for minor surgeries comprising of
1. a central elliptical opening 3″ wide and 4-8″ length,
2. a deep skin surface has adherent areas 2″ wide
as an area around the elliptical opening,
as area of the edges of the smaller ends of the rectangular shaped device

24. The claims are for the sterile surgical skin edge patches of different ranges of lengths, each 1″ in difference, and 3-4″ width, made up of paper, about the thickness of the paper towel, and are supplied in a pouch, with their lengths mentioned on the outside.

25. The device of claim 24 has a superficial surgical surface, and a covered deep adherent skin surface.

26. The device in claims 25, has either straight or concave curvilinear medial edges, the type of which is mentioned out side the pouch, where as their lateral edges are straight in both the types.

27. The claims are for sterile water tanks or cylinders about 10-15 gallons size, transparent, plastic container (disposable but recyclable), that has opening at one place in its cylindrical wall, which is closed by a seal and a cap, and provided with a stop cock.

28. The device of claim 27, is also separately supplied with sterile plastic water faucet in a pouch, which is a small pipe like device, with a 2″ length and 1 cm. internal diameter, that can fit into the opening of the water cylinder, the opening of the tap faucet that fits into the water cylinder, being broader and has threading inside for easy identification or it can be colored differently, and it is also provided with an ‘on and off’ device.

29. A sterile surgical water tank or cylinder with a stop cock, that can be connected to a sterile disposable (recyclable) plastic tubing, to be used for surgical irrigation in the operating room, the end of the tubing having an ‘on and off’ device to be used at the surgical site, and the whole tubing and the ‘on and off’ irrigation device being covered with a sheath that can be removed when the tubing is needed.
